(19) United States
(12) Patent Application Publication
(10) Pub. No.: US 2020/0390371 A1
(43) Pub. Date: Dec. 17, 2020

(54) APPARATUS AND METHOD FOR EVALUATING PHYSICAL ACTIVITY ABILITY

(71) Applicant: ELECTRONICS AND TELECOMMUNICATIONS RESEARCH INSTITUTE, Daejeon (KR)

(72) Inventors: Do-Hyung KIM, Daejeon (KR); Jae-Hong KIM, Daejeon (KR); Jae-Yeon LEE, Daejeon (KR); Min-Su JANG, Daejeon (KR); Sung-Woong SHIN, Daejeon (KR)

(21) Appl. No.: 16/711,328

(22) Filed: Dec. 11, 2019

(30) Foreign Application Priority Data
Jun. 14, 2019 (KR) 10-2019-0070772
Oct. 24, 2019 (KR) 10-2019-0133006

(51) Int. Cl. A61B 5/11; A61B 5/00; A63B 24/00; B25J 9/16

(52) U.S. Cl. CPC A61B 5/1128; A61B 5/0077; A61B 5/1118; A61B 5/1124; A61B 5/7246; A63B 2024/0068; A61B 5/1122; A63B 24/0062; B25J 9/1697; A61B 2576/00; A61B 5/1116

(57) ABSTRACT

Disclosed herein are an apparatus and method for evaluating a physical activity ability. The apparatus includes one or more processors and executable memory for storing at least one program executed by the one or more processors. The at least one program recognizes the position of a human by analyzing an image input through a camera, identifies the motion of the human by analyzing the sequence of the image, and evaluates the physical activity ability of the human from the motion of the human based on the body segment of the human.

### CROSS REFERENCE TO RELATED APPLICATIONS

This application claims the benefit of Korean Patent Application No. 10-2019-0070772, filed Jun. 14, 2019, and Korean Patent Application No. 10-2019-0133006, filed Oct. 24, 2019, which are hereby incorporated by reference in their entireties into this application.

### BACKGROUND OF THE INVENTION

### 1. Technical Field

The present invention relates generally to robot system technology, and more particularly to technology for evaluating the physical activity ability of a human by observing the exercise motion of the human.

### 2. Description of the Related Art

Economic prosperity and improvement of the quality of life lead to rising interest in health and exercise. In order to select an appropriate exercise program, it is necessary for individuals to accurately evaluate their physical activity abilities, and then the individuals may effectively strengthen an aspect of physical fitness in which they are deficient based thereon.

Particularly in the case of children, physical activity ability evaluation results may be used as important criteria for organizing a children's physical education program, which is effective in motor development. Also, in the case of the aged, whose physical functions decline from day to day, it is necessary to continuously monitor their physical activity abilities, and then a personalized exercise program for preventing a decline in physical function may be prescribed based thereon.

Accordingly, the accurate evaluation of a physical activity ability enables active support for preventing a physical function decline.

Methods for evaluating physical activity ability, such as muscular strength, endurance, explosive strength, and the like, are largely categorized into two methods.

In the first method, the subject whose physical activity ability is to be measured performs a physical activity by following a scenario for measuring physical fitness factors, and an expert, specializing in evaluation of a physical activity ability, observes and records the physical activity. However, such a manual method, in which a person evaluates a physical activity ability, has limits on the number of subjects for whom it is possible to measure physical activity ability and has a problem in which the evaluation result may differ depending on the ability, inclination, and subjective view of the expert.

The second method is an automated method in which each physical fitness factor is quantitatively evaluated based on biomechanics information acquired using sensor measurement equipment provided in a professional facility. However, such equipment is an expensive precision system that is used for enhancing the performance of elite players, and is not suitable for use as a device for evaluating physical activity ability that is required in order to help ordinary people live healthy lives.

Accordingly, the present invention proposes a system in which a robot observes the exercise motion of a human and quantitatively evaluates a physical activity ability. The human does not have to visit a certain professional facility and does not have to attach any device to the body. The robot continuously observes/evaluates the physical activity ability of the human, and the human may strengthen the physical fitness factors that he or she lacks depending on the evaluation result. Accordingly, the present invention enables active health support, which helps individuals maintain a healthy life for a long time by preventing a decline in physical function.

This is differentiated from existing passive support, such as simple health care based on the measurement of bio-signals, including blood pressure, pulse, temperature, and the like.

Meanwhile, Korean Patent No. 10-1541095, titled “System for evaluating ability of physical activity”, discloses a method of evaluating the activity ability of a specific body part whereby a user performs a specific task.

### SUMMARY OF THE INVENTION

An object of the present invention is to quantitatively measure various physical fitness factors by analyzing only input image information and to provide an evaluation result.

Another object of the present invention is to present a physical fitness factor that a user lacks based on the result of evaluation of physical activity ability.

A further object of the present invention is to prevent a decline in the physical ability of an individual and to provide active support for helping the individual maintain a healthy life.

In order to accomplish the above objects, an apparatus for evaluating a physical activity ability according to an embodiment of the present invention includes one or more processors; and executable memory for storing at least one program executed by the one or more processors. The at least one program may recognize the position of a human by analyzing an image input through a camera, identify the motion of the human by analyzing the sequence of the image, and evaluate the physical activity ability of the human from the motion of the human based on the body segments of the human.

Here, the at least one program may evaluate the physical activity ability of the human based on the shape of a body segment network, which shows a connection relationship between multiple body segments by connecting the centers of the body segments of the human.

Here, the at least one program may evaluate the posture of the human based on at least one of the position, the orientation, the size, and the shape of the body segment network.

Here, the at least one program may evaluate the motion of the human based on a change in the shape of the body segment network, which is changed depending on the motion of the human.

Here, the at least one program may determine the change in the shape of the body segment network based on the trajectory of the center of the body segment of the human.

Here, the at least one program may evaluate the motion of the human using at least one of the position, the movement range, and the speed of the trajectory.

Here, the at least one program may count the number of repetitions of the motion of the human based on the change in the shape of the body segment network and compare the shape of the body segment network at the start posture of the motion of the human with that at the end posture of the motion of the human, thereby determining whether the motion of the human is finished.

Here, the at least one program may match a motion sequence by comparing the number of images of the motion of the human with the number of images of a standard motion of a previously stored standard motion profile and determine whether the motion of the human is finished based on the degree of motion accuracy of the matched motion sequence.

Here, the at least one program may evaluate at least one of flexibility, endurance, explosive strength, and balance of the human from the result of evaluating the posture of the human and the motion of the human based on the body segment network.

Here, the body segment network may include the torso segment, the multiple arm segments, and the multiple leg segments of the human, and may be formed by connecting the multiple arm segments and the multiple leg segments in the form of a network, with the torso segment as the center.

Here, the body segment network may further include an upper body segment network, in which the upper body center point and the multiple arm segments of the human are connected in the form of a network, and a lower body segment network, in which the lower body center point and the multiple leg segments of the human are connected in the form of a network.

Also, in order to accomplish the above objects, a method for evaluating a physical activity ability, performed by an apparatus for evaluating the physical activity ability, according to an embodiment of the present invention includes recognizing the position of a human by analyzing an image input through a camera; identifying the motion of the human by analyzing the sequence of the image; and evaluating the physical activity ability of the human from the motion of the human based on the body segments of the human.

Here, evaluating the physical activity ability may be configured to evaluate the physical activity ability of the human based on the shape of a body segment network, which shows a connection relationship between multiple body segments by connecting the centers of the body segments of the human.

Here, evaluating the physical activity ability may be configured to evaluate the posture of the human based on at least one of the position, the orientation, the size, and the shape of the body segment network.

Here, evaluating the physical activity ability may be configured to evaluate the motion of the human based on a change in the shape of the body segment network, which is changed depending on the motion of the human.

Here, evaluating the physical activity ability may be configured to determine the change in the shape of the body segment network based on the trajectory of the center of the body segment of the human.

Here, evaluating the physical activity ability may be configured to evaluate the motion of the human using at least one of the position, the movement range, and the speed of the trajectory.

Here, evaluating the physical activity ability may be configured to count the number of repetitions of the motion of the human based on the change in the shape of the body segment network and to compare the shape of the body segment network at the start posture of the motion of the human with that at the end posture of the motion of the human, thereby determining whether the motion of the human is finished.

Here, evaluating the physical activity ability may be configured to match a motion sequence by comparing the number of images of the motion of the human with the number of images of a standard motion of a previously stored standard motion profile and to determine whether the motion of the human is finished based on the degree of motion accuracy of the matched motion sequence.

Here, evaluating the physical activity ability may be configured to evaluate at least one of flexibility, endurance, explosive strength, and balance of the human from the result of evaluating the posture of the human and the motion of the human based on the body segment network.

Here, the body segment network may include the torso segment, the multiple arm segments, and the multiple leg segments of the human, and may be formed by connecting the multiple arm segments and the multiple leg segments in the form of a network, with the torso segment as the center.

Here, the body segment network may further include an upper body segment network, in which the upper body center point and the multiple arm segments of the human are connected in the form of a network, and a lower body segment network, in which the lower body center point and the multiple leg segments of the human are connected in the form of a network.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a block diagram that shows an apparatus for evaluating a physical activity ability according to an embodiment of the present invention;

FIG. 2 is a block diagram that specifically shows an example of the physical activity ability evaluation unit illustrated in FIG. 1;

FIG. 3 is a view that shows a human body segment network according to an embodiment of the present invention;

FIG. 4 is a view that shows the trajectory of body segments in the human body segment network illustrated in FIG. 3;

FIG. 5 is a flowchart that shows a method for evaluating a physical activity ability according to an embodiment of the present invention;

FIG. 6 is a flowchart that specifically shows an example of the step of evaluating a physical activity ability, illustrated in FIG. 5;

FIG. 7 is a flowchart that specifically shows an example of the step of counting a motion, illustrated in FIG. 6;

FIG. 8 is a flowchart that specifically shows an example of the step of determining whether a human motion is finished, illustrated in FIG. 7; and

FIG. 9 is a view that shows a computer system according to an embodiment of the present invention.

### DESCRIPTION OF THE PREFERRED EMBODIMENTS

The present invention will be described in detail below with reference to the accompanying drawings. Repeated descriptions and descriptions of known functions and configurations which have been deemed to unnecessarily obscure the gist of the present invention will be omitted below. The embodiments of the present invention are intended to fully describe the present invention to a person having ordinary knowledge in the art to which the present invention pertains. Accordingly, the shapes, sizes, etc. of components in the drawings may be exaggerated in order to make the description clearer.

Throughout this specification, the terms “comprises” and/or “comprising” and “includes” and/or “including” specify the presence of stated elements but do not preclude the presence or addition of one or more other elements unless otherwise specified.

Hereinafter, a preferred embodiment of the present invention will be described in detail with reference to the accompanying drawings.

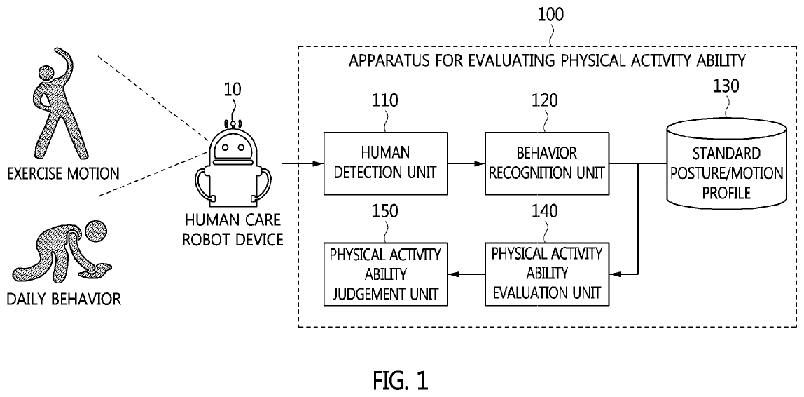

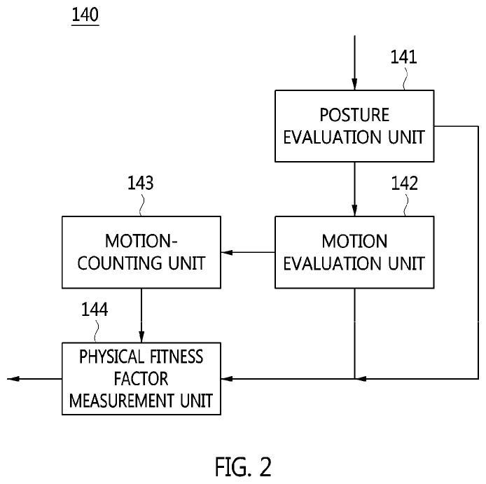

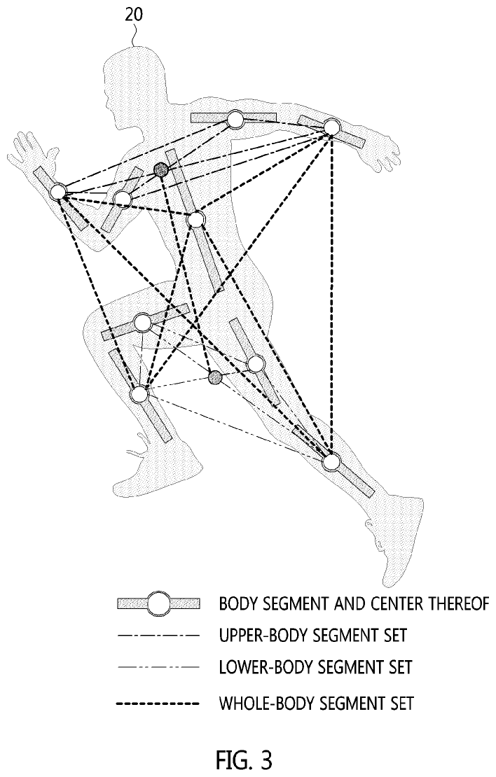

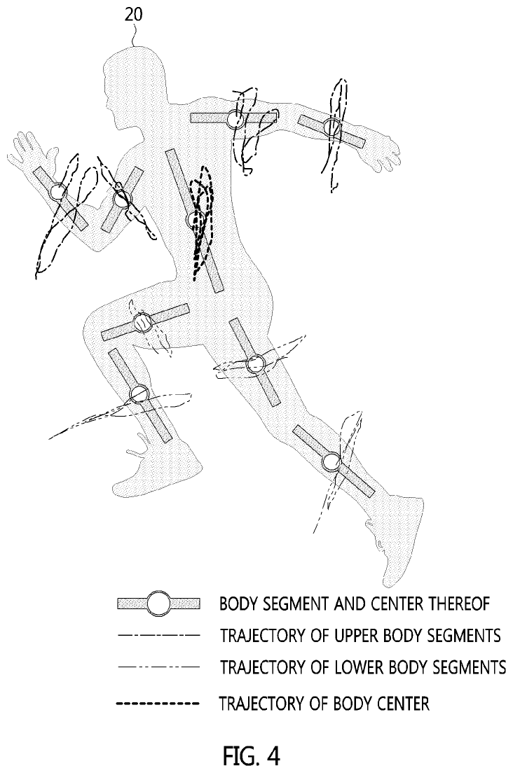

FIG. 1 is a block diagram that shows an apparatus for evaluating a physical activity ability according to an embodiment of the present invention. FIG. 2 is a block diagram that specifically shows an example of the physical activity ability evaluation unit illustrated in FIG. 1. FIG. 3 is a view that shows a human body segment network according to an embodiment of the present invention. FIG. 4 is a view that shows the trajectory of body segments in the human body segment network illustrated in FIG. 3.

Referring to FIG. 1, the apparatus 100 for evaluating a physical activity ability according to an embodiment of the present invention detects a human in an image, which is acquired in such a way that a robot device 10 captures a human exercise motion and daily (usual) behavior using through camera, and evaluates a posture and a motion, thereby evaluating a physical activity ability.

Here, the apparatus 100 for evaluating a physical activity ability may be included in the robot device 10 or may be connected therewith through a wired/wireless network, thereby sharing data therewith.

As shown in FIG. 1, the apparatus 100 for evaluating a physical activity ability according to an embodiment of the present invention includes a human detection unit 110, a behavior recognition unit 120, a standard posture/motion profile storage unit 130, a physical activity ability evaluation unit 140, and a physical activity ability judgement unit 150.

The human detection unit 110 may recognize the position of a human by analyzing an image input through a camera.

Here, the image input to the camera may be an image captured by the camera of the robot device 10 or an image of a human captured by the robot device 10.

Here, the robot device 10 may capture an image of the exercise motion and daily (usual) behavior of the human.

Here, the human detection unit 110 may capture an image of the target whose physical activity ability is to be evaluated using a camera.

Here, the human detection unit 110 may recognize the position of the human in the image, the human corresponding to the target whose physical activity ability is to be evaluated.

Here, the human detection unit 110 may enable the physical activity ability to be evaluated only when a human is recognized in the image, and may switch to a standby state when no human is recognized in the image.

The behavior recognition unit 120 may identify the motion of the human by analyzing the sequence of the image.

For example, in an indoor home environment, the human may perform daily (usual) behavior, such as cleaning, washing the face, having a meal, and the like, or may perform various kinds of indoor exercise for improving health.

Here, the behavior recognition unit 120 may measure and evaluate a physical activity ability from all of the actions taken by the human, or may recognize only a predetermined specific motion, such as an exercise motion of the human, or the like.

A standard posture/motion profile storage unit 130 may store standard posture information and standard motion information for evaluating the physical activity ability of a human.

Here, the exercise motion is a motion that the human makes with the purpose of improving health, and the standard posture/motion profile storage unit 130 may store a standardized posture/motion profile as the standard posture information and the standard motion information. The standard posture/motion profile may be provided in order to facilitate the quantitative measurement and evaluation of a physical activity ability.

Here, upon recognizing that a human is doing a specific type of exercise, the behavior recognition unit 120 may extract the standard posture/motion profile for the corresponding exercise from the standard posture/motion profile storage unit 130.

Here, the behavior recognition unit 120 may input the extracted profile, that is, the standard posture information and the standard motion information, to the physical activity ability evaluation unit 140 along with the image sequence.

The physical activity ability evaluation unit 140 may evaluate the physical activity ability of the human from the motion of the human based on the body segments of the human.

Here, the physical activity ability evaluation unit 140 may evaluate the physical activity ability of the human based on the shape of a body segment network, which shows the connection relationship between multiple body segments by connecting the centers of the body segments of the human.

Referring to FIG. 2, the physical activity ability evaluation unit 140 may include a posture evaluation unit 141, a motion evaluation unit 142, a motion-counting unit 143, and a physical fitness factor measurement unit 144.

When a human makes an exercise motion, the posture evaluation unit 141 compares the posture at a specific time with a standard posture specified in the profile, thereby evaluating the degree of accuracy of the motion.

Here, the posture evaluation unit 141 may evaluate the posture of the human based on at least one of the position, the orientation, the size, and the shape of the body segment network, and may generate posture accuracy evaluation information.

Referring to FIG. 3, a body segment indicates the frame of each body part, and may be a basic element for forming a body segment network.

Here, the body segment network may include the torso segment, the multiple arm segments, and the multiple leg segments of the human, and may be formed by connecting the multiple arm segments and the multiple leg segments in the form of a network, with the torso segment as the center.

Here, the body segment network may further include an upper body segment network, in which the upper body center point and the multiple arm segments of the human are connected in the form of a network, and a lower body segment network, in which the lower body center point and the multiple leg segments of the human are connected in the form of a network.

Here, in the body segment network, nine body segment sets may be used as the whole-body segment set for evaluating the overall posture. The nine body segment sets may include four arm segment sets, four leg segment sets, and a body segment set connected based on an upper-body segment set and a lower-body segment set. Also, in order to evaluate the upper body posture and the lower body posture in detail, six upper-body segment sets and six lower-body segment sets may be further included in the body segment network.

Therefore, each of the posture of the human to be observed and the standard posture may be represented as a body segment network including a total of 21 body segment sets, which include the six upper-body segment sets, the six lower-body segment sets, and the nine whole-body segment sets, and the posture evaluation unit 141 may finally evaluate the degree of accuracy of the overall posture by comparing the position, the orientation, the size, and the shape of the body segment network.

Here, because the body segment network is represented as faces (surfaces), rather than individual body segments, such that the overall posture is evaluated, it may have a characteristic of being robust to errors in detecting large and small joints.

The motion evaluation unit 142 may evaluate a motion for an exercise section including consecutive postures depending on the image sequence.

Here, the motion evaluation unit 142 may evaluate the motion of the human based on a change in the shape of the body segment network, which is changed depending on the motion of the human.

Here, the motion evaluation unit 142 may determine the change in the shape of the body segment network based on the trajectory of the center of the body segment of the human.

Here, the motion evaluation unit 142 may evaluate the motion of the human using at least one of the position, the movement range, and the speed of the trajectory.

That is, the motion evaluation unit 142 may measure the accuracy, the speed, the timing, and the like of the entire exercise motion performed by the human with respect to the standard motion of each exercise.

Here, for the overall evaluation of the motion, the motion evaluation unit 142 may use the posture accuracy evaluation information pertaining to the human, which is calculated by the above-described posture evaluation unit, and the information about the trajectories of the nine body segments illustrated in FIG. 4.

Referring to FIG. 4, the trajectory along which the center of each of the nine body segments moves is illustrated. The information about the trajectory of the body segment may include history information about the movement of a specific body part when the human makes an exercise motion.

Here, the motion evaluation unit 142 may calculate an analysis result, such as the movement position, the movement range, the movement speed/acceleration, and the like of the body segment from the body segment trajectory information, and may finally generate motion accuracy evaluation information for the human motion by combining the analysis result with the posture accuracy evaluation information.

When the human performs a repetitive exercise motion, the motion-counting unit 143 detects whether the motion is finished, thereby counting the number of times the motion is performed.

In the case of a repetitive motion, only when the motion is counted may the motion accuracy evaluation information calculated by the motion evaluation unit 142 be meaningful.

Here, the motion-counting unit 143 counts the number of repetitions of the motion of the human based on a change in the shape of the body segment network and compares the shape of the body segment network at the start posture of the motion of the human with that at the end posture of the motion of the human, thereby determining whether the motion of the human is finished.

Here, the motion-counting unit 143 may match a motion sequence by comparing the number of images of the motion of the human with the number of images of a standard motion of the previously stored standard motion profile and determine whether the motion of the human is finished based on the degree of motion accuracy of the matched motion sequence.

Here, the motion-counting unit 143 may store an image containing the motion of the human, which is input through a camera when the human makes the exercise motion, in a specific motion image buffer.

Here, the motion-counting unit 143 checks whether a number of images of the motion of the human equal to a preset number is accumulated in the buffer, thereby determining whether the number of accumulated images approaches the total number of images of the standard motion.

For example, when the number of accumulated images becomes equal to or greater than 70% of the total number of images of the standard motion, the motion-counting unit 143 determines that the number of accumulated images approaches the total number of images of the standard motion, thereby matching the standard motion sequence with the human motion sequence.

Here, the motion-counting unit 143 may match the human motion sequence stored in the buffer with the standard motion sequence corresponding thereto using a time-series-data-matching algorithm, such as dynamic time warping (DTW) or the like.

Here, the motion-counting unit 143 may determine whether the motion of the human is finished using the matched motion section and the degree of accuracy of the motion, and may calculate information such as the degree of accuracy, the speed, the timing, and the like of the motion along with the number of repetitions of the motion performed by the human when the motion is determined to be finished.

Here, when it is determined that the motion of the human is not finished, the motion-counting unit 143 may store one frame of the next image of the motion of the human in the buffer and repeat the above operations again.

Also, the motion-counting unit 143 may determine whether the motion of the human is finished depending on detailed conditions.

First, the motion-counting unit 143 may analyze the matched motion section and the degree of motion accuracy.

Here, the motion-counting unit 143 may check the degree of motion accuracy when the matched motion section and the degree of motion accuracy are input.

Here, the motion-counting unit 143 may determine whether the degree of motion accuracy is higher than a preset threshold, and may determine that the motion of the human differs from the standard motion when it is determined that the degree of motion accuracy is not higher than the preset threshold.

For example, the case in which the degree of motion accuracy is not higher than the preset threshold corresponds to the case in which the human still performs the motion or in which the human is not able to perform the motion correctly. Accordingly, the motion-counting unit 143 may determine that the motion is not finished.

Here, when the condition related to the degree of motion accuracy is satisfied, the motion-counting unit 143 may check whether the start posture and the end posture of the motion of the human are the same as those of the standard motion.

That is, the motion-counting unit 143 may compare the two start postures with each other and compare the two end postures with each other using the posture accuracy evaluation information.

Here, when it is determined that the two postures are not the same as each other due to the low similarity therebetween, the motion-counting unit 143 may determine that the motion is not completely finished.

Also, when the condition is satisfied because the two postures match each other, the motion-counting unit 143 may compare the movement distance and the movement range of each body segment, caused by the motion of the human, with the movement distance and the movement range of each body segment in the standard motion.

That is, when the motion is performed, the motion-counting unit 143 may check the degree of completeness of the motion in each body segment, rather than checking the overall motion.

Here, the motion-counting unit 143 may compare the respective body segments in the standard motion with the respective body segments in the motion of the human, and may determine that the motion is not completely finished when the difference therebetween is large.

That is, the motion-counting unit 143 may accurately calculate a repetitive motion section using the three conditions described above.

Here, the motion-counting unit 143 may store the motion section satisfying all of the three conditions in a matched motion section history buffer, and may determine that the motion is finally finished when n most recently stored matched motion sections are the same as each other.

Here, the motion-counting unit 143 may determine whether the difference between the movement distance of each body segment and the movement range thereof is less than a threshold, and may determine that the motion is finished when the difference is less than the threshold.

The physical fitness factor measurement unit 144 analyzes the information measured by the posture evaluation unit 141, the motion evaluation unit 142, and the motion-counting unit 143, thereby calculating health-related physical fitness factors, such as muscular strength, flexibility, and the like, and performance-related physical fitness factors, such as speed, balance, and the like.

For example, the physical fitness factor measurement unit 144 may measure physical fitness factors related to flexibility, endurance, explosive strength, and balance.

Here, the physical fitness factor measurement unit 144 may evaluate at least one of the flexibility, the endurance, the explosive strength, and the balance of the human from the result of evaluating the posture and the motion of the human based on the body segment network.

Flexibility is a factor that represents the limit of a range within which a muscle is movable, and the physical fitness factor measurement unit 144 may measure flexibility using the size of the movement range of each body segment, which is provided by the motion evaluation unit 142.

That is, the movement range of the respective body segments in the standard motion is compared with that in the motion of the human, and when the movement ranges match each other or when the movement range in the motion of the human is larger than that in the standard motion, the measured value of the flexibility may have a higher value.

Endurance is a factor that represents the ability to sustain exercise for a long period of time, and the physical fitness factor measurement unit 144 may measure endurance using the number of times a repetitive motion is performed and a trend in the time spent for the repetitive motion, which are provided by the motion-counting unit 143. When the number of times the human performs the repetitive motion is large and when the time spent performing the motion is constant, the endurance may be highly evaluated.

Explosive strength is a factor that represents the ability to exert force instantaneously, and the physical fitness factor measurement unit 144 may measure explosive strength using the movement speed/acceleration of each body segment, which is provided by the motion evaluation unit 142.

Balance is a factor that represents the ability to move in order to maintain equilibrium depending on the situation, and the physical fitness factor measurement unit 144 may measure balance using the body segment network provided by the posture evaluation unit 141. When a motion is compared with a standard motion provided for the purpose of balance measurement, as the degree of posture accuracy is higher, the balance may be regarded as being higher.

The physical fitness factor measurement unit 144 may measure various physical fitness factors in addition to the above-described physical fitness factors, and may evaluate the physical activity ability of the human.

The physical activity ability judgement unit 150 may provide the overall judgement result based on the physical activity ability of the human.

Here, the physical activity ability judgement unit 150 synthetically analyzes the result of evaluation of the physical activity ability including the various physical fitness factors measured by the physical fitness factor measurement unit 144, thereby providing the overall judgement result for the physical activity ability of the human. The result may be provided depending on standardized judgement criteria established by experts specializing in physical activity ability measurement, and a user may effectively strengthen the physical fitness factor that the user lacks through the accurate evaluation of the physical activity ability.

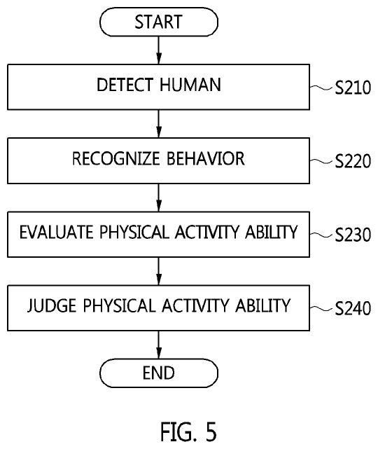

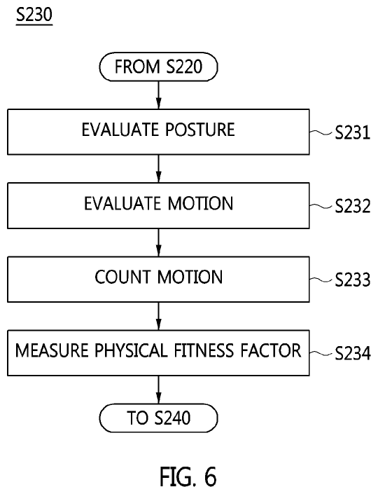

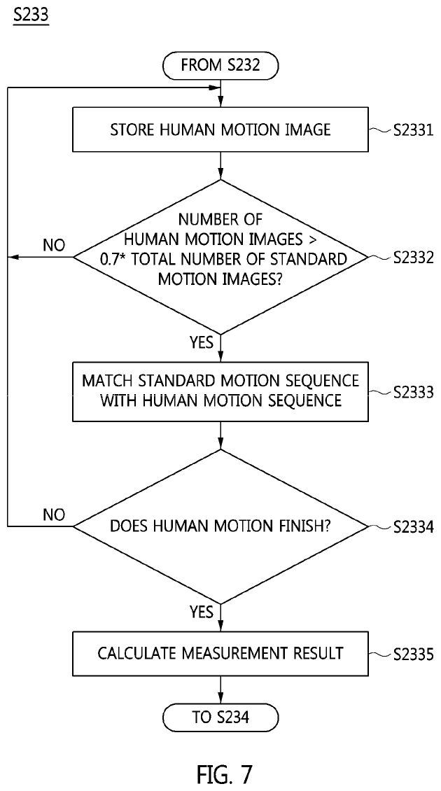

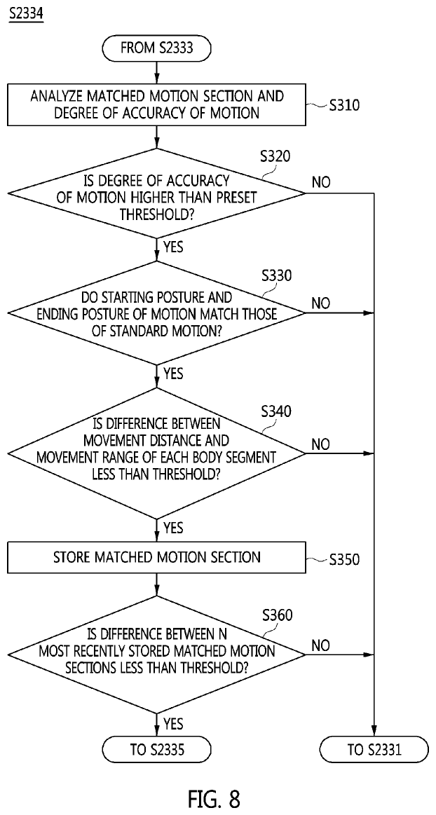

FIG. 5 is a flowchart that shows a method for evaluating a physical activity ability according to an embodiment of the present invention. FIG. 6 is a flowchart that specifically shows an example of the step of evaluating a physical activity ability illustrated in FIG. 5. FIG. 7 is a flowchart that specifically shows an example of the step of counting a motion illustrated in FIG. 6. FIG. 8 is a flowchart that specifically shows an example of the step of determining whether a human motion is finished, illustrated in FIG. 7.

Referring to FIG. 5, in the method for evaluating a physical activity ability according to an embodiment of the present invention, first, a human may be detected in an image at step S210.

That is, at step S210, the image input through a camera is analyzed, whereby the position of the human may be recognized.

Here, the image input through the camera may be an image captured by the camera of a robot device 10 or an image of a human captured by the robot device 10.

Here, at step S210, the robot device 10 may capture an image of the exercise motion and daily (usual) behavior of the human.

Here, at step S210, an image of the target whose physical activity ability is to be evaluated may be captured using a camera.

Here, at step S210, the position of the human in the image may be recognized. Here, the human corresponds to the target whose physical activity ability is to be evaluated.

Here, at step S210, the physical activity ability is evaluated only when a human is detected in the image. When no human is detected in the image, the process may switch to a standby state.

Also, in the method for evaluating a physical activity ability according to an embodiment of the present invention, the behavior of the human may be recognized at step S220.

That is, at step S220, the motion of the human may be identified by analyzing the sequence of the image.

For example, in an indoor home environment, the human may perform daily (usual) behavior, such as cleaning, washing the face, having a meal, and the like, or may perform various kinds of indoor exercise for improving health.

Here, at step S220, the physical activity ability may be measured and evaluated from all of the actions taken by the human, or only a predetermined specific motion, such as an exercise motion of the human, or the like, may be recognized.

Here, at step S220, when it is recognized that the human is doing a specific type of exercise, a standard posture/motion profile for the corresponding exercise may be extracted from the standard posture/motion profile storage unit 130.

Here, at step S220, the physical activity ability may be evaluated using the extracted profile, that is, standard posture information and standard motion information, along with the image sequence.

The standard posture/motion profile storage unit 130 may store the standard posture information and the standard motion information for evaluating the physical activity ability of the human.

Here, the exercise motion is a motion that the human makes with the purpose of improving health, and a standardized posture/motion profile may be stored in the standard posture/motion profile storage unit 130 as the standard posture information and the standard motion information. The standard posture/motion profile may be provided in order to facilitate the quantitative measurement and evaluation of physical activity ability.

Also, in the method for evaluating a physical activity ability according to an embodiment of the present invention, the physical activity ability may be evaluated at step S230.

That is, at step S230, the physical activity ability of the human may be evaluated from the motion of the human based on the body segments of the human.

Here, at step S230, the physical activity ability of the human may be evaluated based on the shape of a body segment network, which shows the connection relationship between multiple body segments by connecting the centers of the body segments of the human.

Referring to FIG. 6, at step S230, first, the posture of the human may be evaluated at step S231.

That is, at step S231, when the human makes an exercise motion, the posture at a specific time is compared with a standard posture specified in the profile, whereby the degree of accuracy of the motion may be evaluated.

Here, at step S231, the posture of the human may be evaluated based on at least one of the position, the orientation, the size, and the shape of the body segment network, whereby posture accuracy evaluation information may be generated.

Referring to FIG. 3, a body segment indicates the frame of each body part, and may be a basic element for forming a body segment network.

Here, the body segment network may include the torso segment, the multiple arm segments, and the multiple leg segments of the human, and may be formed by connecting the multiple arm segments and the multiple leg segments in the form of a network, with the torso segment as the center.

Here, the body segment network may further include an upper body segment network, in which the upper body center point and the multiple arm segments of the human are connected in the form of a network, and a lower body segment network, in which the lower body center point and the multiple leg segments of the human are connected in the form of a network.

Here, in the body segment network, nine body segment sets may be used as the whole-body segment set for evaluating the overall posture. The nine body segment sets may include four arm segment sets, four leg segment sets, and a body segment set connected based on an upper-body segment set and a lower-body segment set. Also, in order to evaluate the upper body posture and the lower body posture in detail, six upper-body segment sets and six lower-body segment sets may be further included in the body segment network.

Therefore, each of the posture of the human to be observed and the standard posture may be represented as a body segment network including a total of 21 body segment sets, which include the six upper-body segment sets, the six lower-body segment sets, and the nine whole-body segment sets, and the posture evaluation unit 141 may finally evaluate the degree of accuracy of the overall posture by comparing the position, the orientation, the size, and the shape of the body segment network.

Here, because the body segment network is represented as faces (surfaces), rather than individual body segments (lines), such that the overall posture is evaluated, it may have a characteristic of being robust to errors in detecting large and small joints.

Also, at step S230, the motion of the human may be evaluated at step S232.

That is, at step S232, the motion for an exercise section including consecutive postures may be evaluated depending on the image sequence.

Here, at step S232, the motion of the human may be evaluated based on a change in the shape of the body segment network, which is changed depending on the motion of the human.

Here, at step S232, the change in the shape of the body segment network may be determined based on the trajectory of the center of the body segment of the human.

Here, at step S232, the motion of the human may be evaluated using at least one of the position, the movement range, and the speed of the trajectory.

That is, at step S232, the accuracy, the speed, the timing, and the like of the entire exercise motion performed by the human may be measured with respect to the standard motion of each exercise.

Here, at step S232, the posture accuracy evaluation information pertaining to the human, which is calculated by the above-described posture evaluation unit, and the information about the trajectories of the nine body segments illustrated in FIG. 4 may be used for the overall evaluation of the motion.

Referring to FIG. 4, the trajectory along which the center of each of the nine body segments moves is illustrated. The information about the trajectory of the body segment may include history information about the movement of a specific body part when the human makes an exercise motion.

Here, at step S232, analysis results, such as the movement position, the movement range, the movement speed/acceleration, and the like of the body segment, may be calculated from the body segment trajectory information, and the analysis results are combined with the posture accuracy evaluation information, whereby motion accuracy evaluation information for the motion of the human may be finally generated.

Also, at step S230, motion counting may be performed at step S233.

That is, at step S233, when the human performs a repetitive exercise motion, whether the motion is finished is detected, whereby the number of times the motion is performed may be counted.

In the case of a repetitive motion, only when the motion is counted may the motion accuracy evaluation information calculated by the motion evaluation unit 142 be meaningful.

Here, at step S233, the number of repetitions of the motion of the human is counted based on a change in the shape of the body segment network, and the shape of the body segment network at the start posture of the motion of the human is compared with that at the end posture of the motion of the human, whereby whether the motion of the human is finished may be determined.

Here, at step S233, a motion sequence is matched by comparing the number of images of the motion of the human with the number of images of a standard motion of a previously stored standard motion profile, and whether the motion of the human is finished may be determined based on the degree of motion accuracy of the matched motion sequence.

Referring to FIG. 7, at step S233, first, an image of the motion of the human may be stored at step S2331.

That is, at step S2331, an image of the motion of the human, which is input through a camera when the human makes the exercise motion, may be stored in a specific motion image buffer.

Also, at step S223, the number of images of the motion of the human may be compared with the total number of images of a standard motion at step S2332.

Here, at step S2332, whether a number of images of the motion of the human equal to a preset number is accumulated in the buffer is checked, whereby whether the number of accumulated images approaches the total number of images of the standard motion may be determined.

For example, at step S2332, when the number of accumulated images becomes equal to or greater than 70% of the total number of images of the standard motion, it is determined that the number of accumulated images approaches the total number of images of the standard motion, and the standard motion sequence may be matched with the human motion sequence at step S2333.

Here, at step S2333, the human motion sequence stored in the buffer may be matched with the standard motion sequence corresponding thereto using a time-series-data-matching algorithm, such as dynamic time warping (DTW) or the like.

Also, at step S233, whether the motion of the human is finished may be determined at step S2334.

Here, at step S2334, whether the motion of the human is finished may be determined using the matched motion section and the degree of motion accuracy, and when the motion is determined to be finished, information such as the degree of accuracy, the speed, the timing, and the like of the motion may be calculated at step S2335 along with the number of repetitions of the motion performed by the human.

Here, at step S2334, when it is determined that the motion of the human is not finished, one frame of the next image of the motion of the human is stored in the buffer, and the process goes back to step S2331, whereby the series of operations in step S233 may be repeated.

Also, at step S2334, whether the motion of the human is finished may be determined depending on detailed conditions.

Referring to FIG. 8, at step S2334, first, the matched motion section and the degree of motion accuracy may be analyzed at step S310.

That is, at step S310, when the matched motion section and the degree of motion accuracy are input, the degree of motion accuracy may be checked at step S320.

Here, at step S320, whether the degree of motion accuracy is higher than a preset threshold is determined. When the degree of motion accuracy is higher than the preset threshold, it is determined that the motion of the human is similar to the standard motion at step S330. When the degree of motion accuracy is not higher than the preset threshold, it is determined that the motion of the human differs from the standard motion, and thus the process may go back to step S2331.

For example, the case in which the degree of motion accuracy is not higher than the preset threshold at step S320 corresponds to the case in which the human still performs the motion or in which the human is not able to perform the motion correctly. Therefore, it may be determined that the motion is not finished.

Also, at step S2334, when the condition related to the degree of motion accuracy is satisfied, whether the start posture and the end posture of the motion of the human are the same as those of the standard motion may be determined at step S330.

That is, at step S330, using the posture accuracy evaluation information, the two start postures may be compared with each other and the two end postures may be compared with each other.

Here, at step S330, when it is determined that the two postures are not the same as each other due to the low similarity therebetween, it is determined that the motion is not completely finished, and the process may go back to step S2331.

Also, at step S2334, when the condition is satisfied because the two postures match each other, the movement distance and the movement range of each body segment, caused by the motion of the human, may be compared with the movement distance and the movement range of each body segment in the standard motion at step S340.

That is, at step S340, when the motion is performed, the degree of completeness of the motion may be checked for each body segment, rather than for the overall motion.

Here, at step S340, the respective body segments of the standard motion are compared with the respective body segments of the motion of the human, and when the difference therebetween is large, it is determined that the motion is not completely finished, and the process may go back to step S2331.

That is, at step S2334, the accurate repetitive motion section may be calculated through the three conditions described above.

Here, at step S2334, the motion section satisfying all of the three conditions is stored in the matched motion section history buffer at step S350, and whether n most recently stored matched motion sections are the same as each other is determined at step S360, whereby whether the motion is finally finished may be determined.

Here, at step S360, whether the difference between the movement distance of each body segment and the movement range thereof is less than a threshold is determined, and when the difference is less than the threshold, it may be finally determined that the motion is finished. In this case, if the n most recently stored matched motion sections have differences (accuracy, position, movement distance and/or movement range) less than a predetermined value, it may be finally determined that the motion is finished.

Also, at step S230, the physical fitness factors of the human are measured from the results of performing posture evaluation, motion evaluation, and motion counting, and the physical ability may be evaluated at step S234.

That is, at step S234, the information measured at steps S231, S232 and S233 is analyzed, whereby health-related physical fitness factors, such as muscular strength, flexibility, and the like, and performance-related physical fitness factors, such as speed, balance, and the like, may be calculated.

For example, at step S234, physical fitness factors related to flexibility, endurance, explosive strength, and balance may be measured.

Here, at step S234, at least one of the flexibility, the endurance, the explosive strength, and the balance of the human may be evaluated from the result of evaluating the posture and the motion of the human based on the body segment network.

Flexibility is a factor that represents the limit of a range within which a muscle is movable, and may be measured at step S234 using the size of the movement range of the respective body segments, which is provided at step S232.

That is, the movement range of the respective body segments in the standard motion is compared with that in the motion of the human, and when the movement ranges match each other or when the movement range in the motion of the human is larger than that in the standard motion, the measured value of the flexibility may have a higher value.

Endurance is a factor that represents the ability to sustain exercise for a long period of time, and may be measured at step S234 using the number of times a repetitive motion is performed and a trend in the time spent for the repetitive motion, which are provided at step S233. When the number of times the human performs the repetitive motion is large and when the time spent performing the motion is constant, the endurance may be highly evaluated.

Explosive strength is a factor that represents the ability to exert force instantaneously, and may be measured at step S234 using the movement speed/acceleration of each body segment, which is provided at step S232.

Balance is a factor that represents the ability to move in order to maintain equilibrium depending on the situation, and may be measured at step S234 using the body segment network provided at step S231. When a motion is compared with a standard motion provided for the purpose of balance measurement, as the degree of posture accuracy is higher, the balance may be regarded as being higher.

In addition to the above-described physical fitness factors, various physical fitness factors may be measured at step S234, and the physical activity ability of the human may be evaluated.

Also, in the method for evaluating a physical activity ability according to an embodiment of the present invention, the physical activity ability may be judged at step S240.

That is, at step S240, the overall judgement result based on the physical activity ability of the human may be provided

Here, at step S240, the overall judgement result for the physical activity ability of the human may be provided by synthetically analyzing the result of evaluation of the physical activity ability including the various physical fitness factors measured at step S234. The result may be provided depending on standardized judgement criteria established by experts specializing in physical activity ability measurement, and a user may effectively strengthen an aspect of physical fitness in which the user is deficient through accurate evaluation of the physical activity ability.

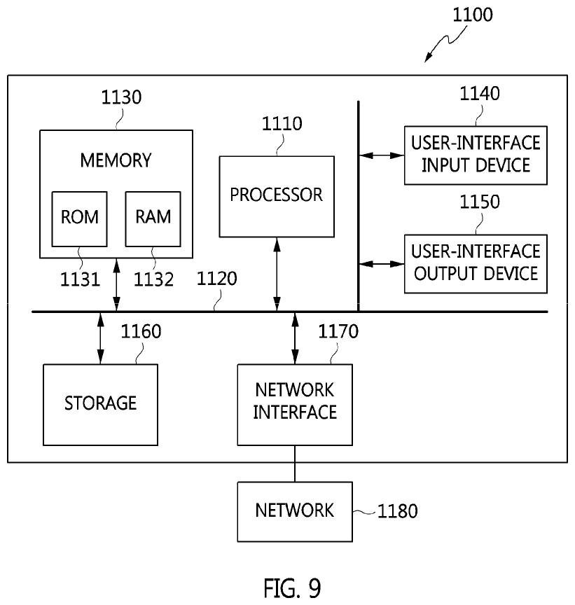

FIG. 9 is a view that shows a computer system according to an embodiment of the present invention.

Referring to FIG. 9, the apparatus 100 for evaluating a physical activity ability according to an embodiment of the present invention may be implemented in a computer system 1100 including a computer-readable recording medium. As shown in FIG. 9, the computer system 1100 may include one or more processors 1110, memory 1130, a user-interface input device 1140, a user-interface output device 1150, and storage 1160, which communicate with each other via a bus 1120. Also, the computer system 1100 may further include a network interface 1170 connected to a network 1180. The processor 1110 may be a central processing unit or a semiconductor device for executing processing instructions stored in the memory 1130 or the storage 1160. The memory 1130 and the storage 1160 may be any of various types of volatile or nonvolatile storage media. For example, the memory may include ROM 1131 or RAM 1132.

The apparatus 100 for evaluating a physical activity ability according to an embodiment of the present invention includes one or more processors 1110 and executable memory 1130 for storing at least one program executed by the one or more processors. The at least one program may recognize the position of a human by analyzing an image input through a camera, identify the motion of the human by analyzing the sequence of the image, and evaluate the physical activity ability of the human from the motion of the human based on the body segments of the human.

Here, at least one program may evaluate the physical activity ability of the human based on the shape of a body segment network, which shows the connection relationship between multiple body segments by connecting the centers of the body segments of the human.

Here, the at least one program may evaluate the posture of the human based on at least one of the position, the orientation, the size, and the shape of the body segment network.

Here, the at least one program may evaluate the motion of the human based on a change in the shape of the body segment network, which is changed depending on the motion of the human.

Here, the at least one program may determine the change in the shape of the body segment network based on the trajectory of the center of the body segment of the human.

Here, the at least one program may evaluate the motion of the human using at least one of the position, the movement range, and the speed of the trajectory.

Here, the at least one program may count the number of repetitions of the motion of the human based on the change in the shape of the body segment network, and may determine whether the motion of the human is finished by comparing the shape of the body segment network at the start posture of the motion of the human with that at the end posture of the motion of the human.

Here, the at least one program may match the motion sequence by comparing the number of images of the motion of the human with the number of images of the standard motion of a previously stored standard motion profile, and may determine whether the motion of the human is finished based on the degree of motion accuracy of the matched motion sequence.

Here, the at least one program may evaluate at least one of the flexibility, the endurance, the explosive strength, and the balance of the human from the result of evaluating the posture of the human and the motion of the human based on the body segment network.

Here, the body segment network may include the torso segment, the multiple arm segments, and the multiple leg segments of the human, and may be formed by connecting the multiple arm segments and the multiple leg segments in the form of a network, with the torso segment as the center.

Here, the body segment network may further include an upper body segment network, in which the upper body center point and the multiple arm segments of the human are connected in the form of a network, and a lower body segment network, in which the lower body center point and the multiple leg segments of the human are connected in the form of a network.

The present invention may quantitatively measure various physical fitness factors by analyzing only input image information, and may provide an evaluation result.

Also, the present invention may indicate an aspect of physical fitness in which a user is deficient based on a result of evaluation of a physical activity ability.

Also, the present invention may prevent a decline of the physical ability of an individual and provide active support for helping the individual maintain a healthy life.

As described above, the apparatus and method for evaluating a physical activity ability according to the present invention are not limitedly applied to the configurations and operations of the above-described embodiments, but all or some of the embodiments may be selectively combined and configured, so that the embodiments may be modified in various ways.

What is claimed is:

1. An apparatus for evaluating a physical activity ability, comprising:
one or more processors; and
executable memory for storing at least one program executed by the one or more processors,
wherein the at least one program recognizes a position of a human by analyzing an image input through a camera, identifies a motion of the human by analyzing a sequence of the image, and evaluates a physical activity ability of the human from the motion of the human based on body segments of the human.

2. The apparatus of claim 1, wherein the at least one program evaluates the physical activity ability of the human based on a shape of a body segment network, which shows a connection relationship between multiple body segments by connecting centers of the body segments of the human.

3. The apparatus of claim 2, wherein the at least one program evaluates a posture of the human based on at least one of a position, an orientation, a size, and the shape of the body segment network.

4. The apparatus of claim 2, wherein the at least one program evaluates the motion of the human based on a change in the shape of the body segment network, which is changed depending on the motion of the human.

5. The apparatus of claim 4, wherein the at least one program determines the change in the shape of the body segment network based on a trajectory of the center of the body segment of the human.

6. The apparatus of claim 5, wherein the at least one program evaluates the motion of the human using at least one of a position, a movement range, and a speed of the trajectory.

7. The apparatus of claim 5, wherein the at least one program counts a number of repetitions of the motion of the human based on the change in the shape of the body segment network and compares the shape of the body segment network at a start posture of the motion of the human with that at an end posture of the motion of the human, thereby determining whether the motion of the human is finished.

8. The apparatus of claim 6, wherein the at least one program matches a motion sequence by comparing a number of images of the motion of the human with a number of images of a standard motion of a previously stored standard motion profile and determines whether the motion of the human is finished based on a degree of motion accuracy of the matched motion sequence.

9. The apparatus of claim 2, wherein the body segment network includes a torso segment, multiple arm segments, and multiple leg segments of the human, and is formed by connecting the multiple arm segments and the multiple leg segments in a form of a network with the torso segment as a center.

10. The apparatus of claim 9, wherein the body segment network further includes:
an upper body segment network in which an upper body center point and the multiple arm segments of the human are connected in a form of a network; and
a lower body segment network in which a lower body center point and the multiple leg segments of the human are connected in a form of a network.

11. A method for evaluating a physical activity ability, performed by an apparatus for evaluating the physical activity ability, comprising:
recognizing a position of a human by analyzing an image input through a camera;
identifying a motion of the human by analyzing a sequence of the image; and
evaluating a physical activity ability of the human from the motion of the human based on body segments of the human.

12. The method of claim 11, wherein evaluating the physical activity ability is configured to evaluate the physical activity ability of the human based on a shape of a body segment network, which shows a connection relationship between multiple body segments by connecting centers of the body segments of the human.

13. The method of claim 12, wherein evaluating the physical activity ability is configured to evaluate a posture of the human based on at least one of a position, an orientation, a size, and the shape of the body segment network.

14. The method of claim 12, wherein evaluating the physical activity ability is configured to evaluate the motion of the human based on a change in the shape of the body segment network, which is changed depending on the motion of the human.

15. The method of claim 14, wherein evaluating the physical activity ability is configured to determine the change in the shape of the body segment network based on a trajectory of the center of the body segment of the human.

16. The method of claim 15, wherein evaluating the physical activity ability is configured to evaluate the motion of the human using at least one of a position, a movement range, and a speed of the trajectory.

17. The method of claim 15, wherein evaluating the physical activity ability is configured to count a number of repetitions of the motion of the human based on the change in the shape of the body segment network and to compare the shape of the body segment network at a start posture of the motion of the human with that at an end posture of the motion of the human, thereby determining whether the motion of the human is finished.

18. The method of claim 16, wherein evaluating the physical activity ability is configured to match a motion sequence by comparing a number of images of the motion of the human with a number of images of a standard motion of a previously stored standard motion profile and to determine whether the motion of the human is finished based on a degree of motion accuracy of the matched motion sequence.

19. The method of claim 12, wherein the body segment network includes a torso segment, multiple arm segments, and multiple leg segments of the human, and is formed by connecting the multiple arm segments and the multiple leg segments in a form of a network with the torso segment as a center.

20. The method of claim 19, wherein the body segment network further includes:
an upper body segment network in which an upper body center point and the multiple arm segments of the human are connected in a form of a network; and
a lower body segment network in which a lower body center point and the multiple leg segments of the human are connected in a form of a network.
